(19) United States
(12) Patent Application Publication
(10) Pub. No.: US 2021/0129111 A1
(43) Pub. Date: May 6, 2021

(54) COMPOSITE SORBENT, DEVICES, AND METHODS

(71) Applicant: SABIC GLOBAL TECHNOLOGIES B.V., BERGEN OP ZOOM (NL)

(72) Inventor: Dmytro Tymoshenko, Albany, NY (US)

(21) Appl. No.: 16/629,504

(22) PCT Filed: Jul. 25, 2018
(86) PCT No.: PCT/US2018/043707

§ 371 Date: Jan. 8, 2020

Related U.S. Application Data
(60) Provisional application No. 62/537,217, filed on Jul. 26, 2017.

(51) Int. Cl. B01J 20/26; B01J 20/22; B01J 20/10; B01J 20/28; B01J 20/32; B01J 20/34; A61M 1/16; A61M 1/36; A61M 1/34; G01N 1/40; B01D 15/20

(52) U.S. Cl. CPC B01J 20/267; A61M 2202/0498; B01J 20/22; B01J 20/103; B01J 20/2808; B01J 20/28083; B01J 20/28085; B01J 20/28071; B01J 20/28073; B01J 20/28076; B01J 20/28059; B01J 20/28061; B01J 20/28064; B01J 20/28066; B01J 20/28021; B01J 20/3293; B01J 20/321; B01J 20/3278; B01J 20/3219; B01J 20/3475; B01J 20/3425; A61M 1/1696; A61M 1/3679; A61M 1/3403; A61M 1/1607; G01N 1/405; B01D 15/203; A61M 2230/20; B01J 20/261

(57) ABSTRACT

A composite sorbent composition comprising a polymeric adsorbent; and an extractant having the formula (I), or hydrate in thereof, wherein X is O or S, A1 and A2 are each independently —C(O)— or —C(R′)(R″)— wherein R′, and R″ are each independently hydrogen, halogen, hydroxyl, cyano, nitro, amino, —CHO, —COOH, C1-12 alkyl, C1-4 alkoxy, C1-4 alkylamino, C1-2 haloalkyl, C1-2 haloalkoxy, C1-12 cycloalkyl, C6-12 aryl, C7-13 arylalkyl, C3-12 heteroaryl, C1-12 heteroalkyl, or C4-12 heteroarylalkyl, Z is a covalent bond, —S—, —O—, —SO2—, —SO—, —P(R)(═O)—, —NR—, -C(O)-, -C(O)NH-, —C(═N—R)—, or —C(R′)(R″)— wherein R, R′, and R″ are each independently hydrogen, halogen, hydroxyl, cyano, nitro, amino, —CHO, —COOH, —C(O)NH2, C1-12 alkyl, C1-12 alkoxy, C1-12 alkylamino, C1-4 haloalkyl, C1-4 haloalkoxy, C4-12 cycloalkyl, C6-12 aryl, C7-13 arylalkyl, C3-12 heterocycloalkyl, C3-12 heteroaryl, C1-12 heteroalkyl, or C4-12 heteroarylalkyl, and R1 and R2 are each independently hydrogen, halogen, hydroxyl, cyano, nitro, amino, or a substituted or unsubstituted monovalent C1-40 hydrocarbon.

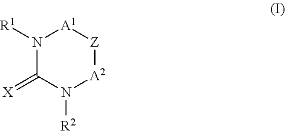

### BACKGROUND

Solid phase extraction (SPE) of nitrogen-containing compounds can be used for extraction of such compounds from their aqueous and organic solutions for purification, isolation, or detection in industrial applications, medical applications, environmental protection, and biotechnology. For example, SPE technology is used in medical applications for the sorption of urea, creatinine, and similar nitrogen containing compounds in dialysates, i.e., aqueous solutions resulting from hemodialysis treatments.

SPE separation methods are applicable to a variety of organic compounds of different polarity and basicity or acidity. However, SPE is rarely used to isolate highly polar or low-basicity compounds (or both), such as amides and ureas. Sorbents for these types of adsorbates (also known as sorbates) have commonly required multistep synthesis to achieve increased sorbent-sorbate affinity. Sorbent systems for urea adsorbates suffer from several problems, including higher cost and lower safety in biological or medicinal applications.

Thus, there remains a need for new sorbents that are lower cost. It would be a further advantage if the sorbents demonstrate desirable sorption efficiency. It would be a still further advantage if the sorbents had improved safety for biological, environmental, industrial, medical, or other applications.

### BRIEF DESCRIPTION

In an embodiment, a composite sorbent composition comprises a polymeric adsorbent; and an extractant having the formula

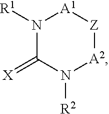

or a hydrate thereof, wherein X is O or S, A¹ and A² are each independently —C(O)— or —C(R′)(R″)— wherein R′, and R″ are each independently hydrogen, halogen, hydroxyl, cyano, nitro, amino, —CHO, —COOH, C₁₋₁₂ alkyl, C₁₋₄ alkoxy, C₁₋₄ alkylamino, C₁₋₂ haloalkyl, C₁₋₂ haloalkoxy, C₁₋₁₂ cycloalkyl, C₆₋₁₂ aryl, C₇₋₁₃ arylalkyl, C₃₋₁₂ heteroaryl, C₁₋₁₂ heteroalkyl, or C₄₋₁₂ heteroarylalkyl, Z is a covalent bond, —S—, —O—, —SO₂—, —SO—, —P(R)(═O)—, —NR—, —C(O)—, —C(O)NH—, —C(═N—R)—, or —C(R′)(R″)— wherein R, R′, and R″ are each independently hydrogen, halogen, hydroxyl, cyano, nitro, amino, —CHO, —COOH, —C(O)NH₂, C₁₋₁₂ alkyl, C₁₋₁₂ alkoxy, C₁₋₁₂ alkylamino, C₁₋₄ haloalkyl, C₁₋₄ haloalkoxy, C₄₋₁₂ cycloalkyl, C₆₋₁₂ aryl, C₇₋₁₃ arylalkyl, C₃₋₁₂ heterocycloalkyl, C₃₋₁₂ heteroaryl, C₁₋₁₂ heteroalkyl, or C₄₋₁₂ heteroarylalkyl, and R¹ and R² are each independently hydrogen, halogen, hydroxyl, cyano, nitro, amino, or a substituted or unsubstituted monovalent C₁₋₄₀ hydrocarbon.

In another embodiment, a method for the manufacture of the composite sorbent composition comprises contacting the extractant and the polymeric adsorbent in a solvent under conditions effective to provide the composition.

In yet another embodiment, a device comprises the composite sorbent composition.

In still another embodiment, a method for separating an analyte from a solution, the method comprises contacting the composite sorbent composition with the solution to form an analyte-bound composition; and separating the solution from the analyte-bound composition to provide a regenerated solution, wherein the amount of the analyte in the regenerated solution is less than the amount of the analyte in the solution.

In another embodiment, a hemodialysis or hemofiltration system is provided for using the method for separating an analyte from a solution, wherein the solution is a dialysate and wherein the analyte is urea, creatinine, uremic acid, or a combination comprising at least one of the foregoing.

The above described and other features are exemplified by the following detailed description, examples, and claims.

### DETAILED DESCRIPTION

This disclosure relates to a composite sorbent composition comprising a polymer adsorbent having a defined porous structure and surface that is surface-modified with an extractant to form two-component composite sorbent. The composite sorbent composition is particularly useful for the treatment of solutions containing sorbates of high polarity, low basicity, or both. In addition, the composite sorbent compositions can be easily obtained, and thus are of lower cost. The sorbents can further demonstrate desirable sorption efficiency. Still further, the sorbents can have improved safety in biological, environmental, industrial, medical, or other applications.

The composite sorbent composition comprises a polymeric adsorbent; and an extractant having the formula (1), or a hydrate thereof:

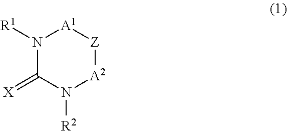

wherein X is O or S. In an embodiment, X is O.

In formula (1), A¹ and A² are each independently —C(O)— or —C(R′)(R″)— wherein R′, and R″ are each independently hydrogen, halogen, hydroxyl, cyano, nitro, amino, —CHO, —COOH, C₁₋₁₂ alkyl, C₁₋₄ alkoxy, C₁₋₄ alkylamino, C₁₋₂ haloalkyl, C₁₋₂ haloalkoxy, C₁₋₁₂ cycloalkyl, C₆₋₁₂ aryl, C₇₋₁₃ arylalkyl, C₃₋₁₂ heteroaryl, C₁₋₂ heteroalkyl, or C₄₋₁₂ heteroarylalkyl.

In formula (1), Z is a covalent bond, —S—, —O—, —SO₂—, —SO—, —P(R)(═O)—, —NR—, —C(O)—, —C(O)NH—, —C(═N—R)—, or —C(R′)(R″)— wherein R, R′, and R″ are each independently hydrogen, halogen, hydroxyl, cyano, nitro, amino, —CHO, —COOH, —C(O)NH₂, C₁₋₁₂ alkyl, C₁₋₁₂ alkoxy, C₁₋₁₂ alkylamino, C₁₋₄ haloalkyl, C₁₋₄ haloalkoxy, C₄₋₁₂ cycloalkyl, C₆₋₁₂ aryl, C₇₋₁₃ arylalkyl, C₃₋₁₂ heterocycloalkyl, C₃₋₁₂ heteroaryl, C₁₋₁₂ heteroalkyl, or C₄₋₁₂ heteroarylalkyl. In an embodiment, Z is —C(R′)(R″)— wherein R′ and R″ are each independently hydrogen, halogen, hydroxyl, amino, C₁₋₄ alkyl, C₁₋₄ alkoxy, C₁₋₄ alkylamino, C₁₋₆ alkylthio, C₁₋₆ alkylsulfonyl, trifluoromethyl, difluoromethyl, or trifluoromethoxy. In another embodiment, Z is —C(O)—.

In formula (1), R¹ and R² are each independently hydrogen, halogen, hydroxyl, cyano, nitro, amino, or a substituted or unsubstituted monovalent C₁₋₄₀ hydrocarbon. In an embodiment, R¹ and R² are each independently hydrogen, C₁₋₁₂ alkyl, C₂₋₁₂ alkenyl, C₂₋₁₂ alkynyl, C₄₋₁₂ cycloalkyl, C₆₋₁₂ aryl, C₇₋₁₃ arylalkyl, C₃₋₁₂ heterocycloalkyl, C₃₋₁₂ heteroaryl, C₁₋₁₂ heteroalkyl, or C₄₋₁₂ heteroarylalkyl, each of which R¹ and R² is unsubstituted or substituted with one or more of halogen, hydroxyl, cyano, nitro, amino, —CHO, —COOH, —C(O)NH₂, C₁₋₁₂ alkyl, C₂₋₁₂ alkenyl, C₂₋₁₂ alkynyl, C₁₋₁₂ alkoxy, C₂₋₁₂ alkanoyl, mono- or di(C₁₋₁₂ alkylamino) C₀₋₈ alkyl, (C₁₋₁₂ alkyl) carboxamide, (C₁₋₁₂ alkyl) ester, C₁₋₁₂ heteroalkyl, C₁₋₄ haloalkyl, or C₁₋₄ haloalkoxy. In another embodiment, R¹ and R² are the same, and are hydrogen or C₁₋₆ alkyl.

In an embodiment, the composite sorbent composition includes the extractant of formula (1), wherein R¹ and R² are each hydrogen, X is O, and Z is —C(O)— or —C(R′)(R″)— wherein R′ and R″ are each hydroxyl. For example, the extractant is a heterocyclic polycarbonyl compound (A and B) or a heterocyclic 2,2-dihydroxy-1,3,-dione compound (C), or a combination comprising at least one of the foregoing, represented by the formulas:

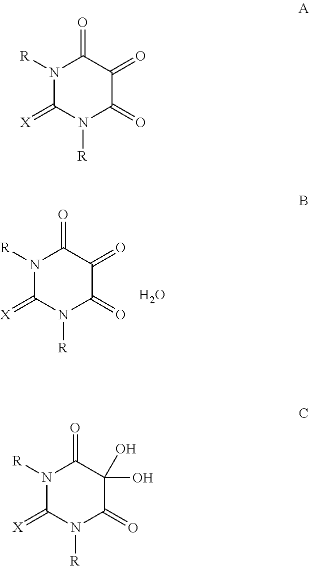

wherein X is O and R¹ and R² are each hydrogen. In an embodiment, the extractant is 2,4,5,6-(1H,3H)-pyrimidinetetrone (alloxan).

The polymeric adsorbent is a polymeric compound with defined surface and porosity characteristics and serves as the scaffold for impregnation with the extractant. As used herein, the “polymeric compound” includes polymers and copolymers. In an embodiment, the polymeric adsorbent is derived from a monomer comprising a vinyl C₆₋₁₂ aryl, a divinyl C₆₋₁₂ aryl, a trivinyl C₆₋₁₂ aryl, a (C₁₋₈ alkyl) (meth)acrylate, an alkylenediol di(meth)acrylate, an alkylenetriol tri(meth)acrylate, a polyester di(meth)acrylate, a (meth)acrylamide, a bis(meth)acrylamide, or a combination comprising at least one of the foregoing. In another embodiment, the polymeric adsorbent comprises poly(styrene-divinylbenzene), sulfonated poly(styrene-divinylbenzene), poly(ethylvinylbenzene-divinylbenzene), poly(amide-divinylbenzene), poly(N-vinylpyrrolidone-divinylbenzene), poly((meth)acrylate-divinylbenzene), poly((meth)acrylonitrile-divinylbenzene), poly(acrylonitrile-divinylbenzene), poly(2-hydroxyethyl (meth)acrylate-ethylstyrene-divinylbenzene), poly(cyanomethylstyrene-divinylbenzene), poly(4-vinylpyridine-divinylbenzene), poly(N-vinylimidazole-divinylbenzene), poly(4-vinylimidazole-divinylbenzene), poly(1-vinyl-2-pyrrolidone-divinylbenzene), poly(para-vinylbenzylchloride-divinylbenzene), poly(metalpara-vinylbenzylchloride-divinylbenzene), poly(2-hydroxyethyl (meth)acrylate-vinylbenzylchloride-divinylbenzene), a poly((C₁₋₈ alkyl) (meth)acrylate), or a combination comprising at least one of the foregoing.

The polymeric adsorbent is not particularly limited, as long as the polymeric compound has a suitable porous structure and surface parameters. Commercially available polymeric adsorbents can be used. For example, poly(styrene-co-divinylbenzene) adsorbents, which are a polystyrene crosslinked with divinylbenzene, are available under the trade name DOWEX OPTIPORE® polymeric adsorbents, sold by The Dow Chemical Company with the designations V-493, V-503, and V-323. Other suitable, poly(styrene-co-divinylbenzene) polymeric adsorbents include those available under the trade names AMBERLITE® polymeric adsorbents, sold by The Dow Chemical Company with the designations FPX62, FPX68, XAD2, XAD4, XAD16HP, XAD18, and XAD1180N; AMBERCHROM™ polymeric adsorbents, sold by The Dow Chemical Company with the designations CG161C, CG161M, CG161S, CG300C, CG300M, CG300S, XT20, and XT30; DIAION™ polymeric adsorbents, sold by Mitsubishi Chemical Corp. with the designations HP20 and HP21; DOSHION™ polymeric adsorbents, sold by Doshi with the designations DAD200, DAD201, DAD300, and DAD301; INDION™ polymeric adsorbents, sold by Ion Exchange India with the designations PA500 and PA800; LEWATIT™ polymeric adsorbents, sold by Lanxess with the designations S7968 and VPOC1064MDPH; MACRONET™ polymeric adsorbents, sold by Purolite with the designations MN200, MN202, MN250, MN252, and MN270; PUROSORB™ polymeric adsorbents, sold by Purolite with the designations PAD350, PAD400, PAD500, PAD550, PAD600, PAD600FM, PAD700, PAD900, and PAD910; RELITE™ polymeric adsorbents, sold by Resindion with the designations SP411 and SP460; SEPABEADS™ polymeric adsorbents, sold by Mitsubishi Chemical Corp. with the designations SP700, SP825L and SP850; and TULSION™ polymeric adsorbents, sold by Thermax with the designations ADS600 and ADS800.

Suitable poly(aliphatic-(meth)acrylate) adsorbents include those available under the trade name AMBERLITE® polymeric adsorbents, sold by The Dow Chemical Company with the designations XAD7HP. Other suitable poly(aliphatic-(meth)acrylate) polymeric adsorbents include, but are not limited to, those available under the trade name PUROSORB™ polymeric adsorbents, sold by Purolite with the designations PAD300, PAD610, and PAD950; DIAION™ polymeric adsorbents, sold by Mitsubishi Chemical Corp. with the designations HP2MG and SP2MGS; SUPELITE™ polymeric adsorbents, sold by Sigma-Aldrich Co. with the designation DAX-8; AMBERCHROM™ polymeric adsorbents, sold by The Dow Chemical Company with the designations CG71C, CG71M, CG71S, and HPS60; TULSION™ polymeric adsorbents, sold by Resin Products Ltd with the designation ADS400; LEWATIT™ polymeric adsorbents, sold by Lanxess with the designation VPOC1600; and RELITE™ polymeric adsorbent, sold by Resindion with the designation SP490.

The polymeric adsorbent has a suitable porous structure and surface, as defined by the average pore diameter, the pore volume, and the specific surface area. The average pore diameter, pore volume, and specific surface area of the polymeric adsorbent are unchanged after the extractant and polymeric adsorbent are contacted to form the composite sorbent composition. In an embodiment, the polymeric adsorbent has an average pore diameter of about 10 to about 1,400 Angstroms (Å), preferably about 12 to about 1,200 Å, more preferably about 14 to about 1,000 Å. The polymeric adsorbent can have an average pore diameter of about 20 to about 900 Å, about 25 to about 800 Å, about 30 to about 700 Å, about 35 to about 600 Å, about 40 to about 500 Å, about 50 to about 400 Å, about 60 to about 350 Å, about 70 to about 300 Å, about 80 to about 250 Å, about 90 to about 200 Å, or about 100 to about 180 Å. In another embodiment, the polymeric adsorbent can have an average pore diameter of about 20 to about 150 Å, about 30 to about 100 Å, or about 40 to about 90 Å.

The polymeric adsorbent can have a pore volume of about 0.1 to about 2.25 milliliters per gram (mL/g), preferably about 0.2 to about 2.0 mL/g, more preferably about 0.3 to about 1.85 mL/g. In an embodiment, the polymeric adsorbent can have a pore volume of about 0.3 to about 1.8 mL/g, about 0.35 to about 1.7 mL/g, about 0.4 to about 1.6 mL/g, about 0.5 to about 1.5 mL/g, about 0.6 to about 1.4 mL/g, about 0.7 to about 1.3 mL/g, or about 0.8 to about 1.2 mL/g. In still another embodiment, the polymeric adsorbent can have a pore volume of about 0.3 to about 1.3 mL/g, about 0.4 to about 1.2 mL/g, or about 0.5 to about 1 mL/g.

The polymeric adsorbent can have a specific surface area of about 50 to about 2,500 square meters per gram (m²/g), preferably about 100 to about 1,800 m²/g, more preferably about 150 to about 1,200 m²/g. In an embodiment, the polymeric adsorbent can have a specific surface area of about 150 to about 1,150 m²/g, about 200 to about 1,100 m²/g, about 300 to about 1,000 m²/g, about 400 to about 900 m²/g, about 450 to about 800 m²/g, or about 500 to about 750 m²/g. In still another embodiment, the polymeric adsorbent can have a specific surface area of about 300 to about 1,200 m²/g, about 400 to about 1,100 m²/g, about 450 to about 1,100 m²/g, about 450 to about 1,000 m²/g, about 500 to about 900 m²/g, or about 600 to about 800 m²/g.

For example, in a specific embodiment, the polymeric adsorbent can have an average pore diameter of about 20 to about 150 Å, about 30 to about 100 Å, or about 40 to about 90 Å; a pore volume of about 0.3 to about 1.3 mL/g, about 0.4 to about 1.2 mL/g, or about 0.5 to about 1 mL/g; and a specific surface area of about 300 to about 1,200 m²/g, about 400 to about 1,100 m²/g, about 450 to about 1,100 m²/g, about 450 to about 1,000 m²/g, about 500 to about 900 m²/g, or about 600 to about 800 m²/g.

The composite sorbent composition can further include an additional protective coating that can be used to form a protective barrier reducing the potential leaching of the extractant from the pores of the composite sorbent into solution. For example, this protective coating can be added in post-impregnation step to form a three-component composite sorbent. As used herein, “a coated composite sorbent” is used interchangeably with “a three-component composite sorbent.”

In an embodiment, the composite sorbent composition can further include a precipitated water-insoluble polymer on the surface of the composite sorbent composition, wherein the water-insoluble polymer is derived from monovinyl aromatic monomers such as styrene, alpha-ethyl styrene, vinyl naphthalene, vinylbenzyl alcohol, or the like; monovinylic monomers such as acrylonitrile, (meth)acrylic acid, vinyl chloride, vinylidene chloride, vinyl formaniide, vinyl C₁₋₆ alkyl ketones, C₁₋₆ alkyl vinyl ethers, a vinyl C₃₋₈ carboxylate such as vinyl acetate, C₁₋₆ alkyl (meth)acrylates, or the like; C₆₋₁₂ aryl sulfones; or a combination comprising at least one of the foregoing; or a cross-linked product of a precipitated water-soluble polymer on the surface of the composition, wherein the precipitated water-soluble polymer comprises poly(vinyl) alcohol; a poly((C₁₋₆ alkyl) hydroxy (meth)acrylate such as poly(hydroxyethyl (meth)acrylate), poly(hydroxypropyl (meth)acrylate), or the like; a hydroxy (C₁₋₆ alkyl) cellulose such as hydroxyethyl cellulose, hydroxypropyl cellulose, or the like; starch; dextrin; an alkali or ammonium acid salt of carboxy(C₁₋₃ alkyl) cellulose ether; a poly(di(C₁₋₆ alkyl)aminoethyl (meth)acrylate) such as poly(dimethylaminoethyl (meth)acrylate), poly(diethylaminoethyl (meth)acrylate), or the like; poly(N-vinylpyrrolidinone); an alkali or ammonium salt of poly(meth)acrylic acid; a poly(meth)acrylamide or partially hydrolyzed derivative thereof; a poly(N-(C₁₋₆ alkyl)(meth)acrylamide) such as poly-N-isopropyl(meth)acrylamide; a poly(N,N-di(C₁₋₆ alkyl)(meth)acrylamide such as poly-N,N-dimethyl(meth)acrylamide; 2-(meth)acrylamido-2-methylpropane sulfonic acid or an alkali salt thereof; or a combination comprising at least one of the foregoing. In another embodiment, the water insoluble polymer is a poly(vinyl (C₁₋₆ alkyl) ketone), a poly(vinyl C₁₋₆ alkyl ether), or poly(C₆₋₁₂ aryl sulfone), preferably a polysulfone, a polyethersulfone, a polyphenylsulfone, or a combination comprising at least one of the foregoing.

According to another aspect, a method for the manufacture of the composite sorbent composition can include contacting the extractant and the polymeric adsorbent in a solvent under conditions effective to provide the composite sorbent composition. The impregnation process includes exposure of the adsorbent polymer to a solution of the extractant in the appropriate solvent. The preparation process can include processes of rinsing of the resulting composite sorbent with another solvent to assure sufficient extractant precipitation on the surface of the adsorbent polymer, rinsing of the resulting composite sorbent composition with a solvent to assure removal of the excess of the extractant from the outer surface of the composite sorbent composition, and drying under reduced or normal pressure using normal or elevated temperatures.

The solvent has no particular limit as long as it can dissolve or disperse the aforementioned components, but can include, for example, at least one of an alcohol solvent such as ethyl alcohol, methyl alcohol, isopropyl alcohol, butanol, or the like; an amide solvent such as dimethylacetamide, dimethylformamide (DMF), or the like; an aqueous solvent such as deionized water; and a mixture of the solvents, but is not limited thereto. In an embodiment, the solvent is deionized water.

Conditions effective to provide the composite sorbent composition can further include temperature and reaction time. In an embodiment, the extractant and the polymeric adsorbent are contacted in the solvent at a temperature of about 25° C. to about 50° C. for about 4 to about 12 hours.

The resulting composite sorbent composition can be filtered and then rinsed with a solvent as described herein. In an embodiment, the composite sorbent composition is rinsed with deionized water. In another embodiment, the composite sorbent composition is dried under reduced pressure at a temperature of about 40 to about 90° C. for a time of about 6 to about 24 hours.

The method can further include precipitating a water-insoluble polymer on the surface of the composite sorbent composition to provide a coated composite sorbent composition. Suitable water-insoluble polymers are as described herein. In an embodiment, the water-insoluble polymer can be contacted with the composite sorbent in a solvent, and the resulting product is filtered, washed, and then dried to obtain the coated composite sorbent composition. In another embodiment, the solvent is a ketone solvent such as methyl isobutyl ketone, 1-methyl-2-pyrrolidinone (NMP), cyclohexanone, acetone, or the like; an ether solvent such as tetrahydrofuran (THF) or methyl tert-butyl ether (MTBE); an ester solvent such as ethyl formate, ethyl acetate, butyl acetate, propylene glycol methyl ether acetate, or the like; carbonate solvent such as dimethyl carbonate, ethylene carbonate, propylene carbonate, or the like; an alcohol solvent such as isopropyl alcohol, butanol, or the like; or an amide solvent such as dimethylacetamide, dimethylformamide (DMF), or the like. In an embodiment, composite sorbent and the water-insoluble polymer can be contacted at a temperature of about 25° C. to about 50° C. for a time of about 20 minutes to about 3 hours.

In another embodiment, the method can include precipitating a water-soluble polymer on a surface of the composite sorbent composition; and cross-linking the water-soluble polymer on the surface to provide the coated composite sorbent composition. The process of cross-linking can be self-crosslinking or reactive, i.e., with participation of a cross-linking reagent. Crosslinking can be catalyzed thermally, photochemically, or chemically. In an embodiment, the water-soluble polymer can be contacted with the composite sorbent in a solvent, and the resulting product can be filtered, washed, and then dried. Subsequently, the coated composite sorbent can be formed by crosslinking, for example at an elevated temperature, in the presence of a suitable catalyst, in the presence of a suitable crosslinking reagent, by exposure to light, heat, or the like.

Suitable catalysts can include zinc salts, sodium borate, boric acid, benzoyl peroxide, citric acid, metal salts of carbonate, such as Cs₂CO₃, NaHCO₃, and Na₂CO₃, or the like. 4

Suitable crosslinking reagents can include divinylbenzene, alkylenediol di(meth)acrylates such as glycol bisacrylate and ethylene glycol dimethacrylate, alkylenetriol tri(meth)acrylates, polyester di(meth)acrylates, bis(meth)acrylamides, triallyl cyanurate, triallyl isocyanurate, allyl (meth)acrylate, diallyl maleate, diallyl fumarate, diallyl adipate, triallyl esters of citric acid, triallyl esters of phosphoric acid, diamines such as tetramethylenediamine, poly(vinyl alcohol), aldehydes such as glutaraldehyde and formaldehyde, epoxy compounds, dialdehydes, or the like, as well as combinations comprising at least one of the foregoing crosslinking reagents.

Thermal crosslinking can be at a temperature of about 80° C. to about 250° C., preferably about 120° C. to about 200° C., for a time of about 5 minutes to about 1 hour, preferably about 10 minutes to about 30 minutes.

According to another aspect, a device comprising a layer of the composite sorbent composition or the coated composite sorbent composition is provided. For example, the layer can be provided in a column or as a cartridge. In an embodiment, the device further includes at least one secondary adsorbent layer, wherein the at least one secondary adsorbent can be activated carbon, silica, modified silica, a second polymeric adsorbent, or a combination comprising at least one of the foregoing. The secondary adsorbent can include a polymer ion exchanger, for example strong cation, strong anion, weak cation, or weak anion exchange resin. Any suitable ion exchange resin, including those available in the art, can be used. The secondary adsorbent can include an inorganic sorbent, for example ion exchange zeolite, zirconium oxide, zirconium phosphate, or nanoclay. Any suitable inorganic sorbent, including those available in the art, can be used.

In an embodiment, the composite sorbent layer or the coated composite sorbent layer of the device can be in an amount of about 5 to about 95 weight percent (wt %), preferably about 10 to about 90 wt %, based on the total adsorbent weight of all adsorbent materials in the device. One or more of the secondary adsorbents can be included as a single layer or multiple layers that are downstream, upstream or both, of the composite sorbent layer along the length of the column (for example, a cartridge). The column diameter to length ratio can be about 1:200 to about 1:1, preferably about 1:20 to about 1:2. Each layer can have a thickness of about 1 to about 1,000 millimeters (mm). A packed column can be prepared by adding the composite sorbent and the one or more secondary adsorbents into the cartridge housing using standard techniques, e.g. loading of dry powder or/and beads, or adding a suspension in the appropriate solvent or solvent mixtures. The chromatographic column can then be ultrasonicated or pressure may be applied to the packing material to improve the uniformity of each layer and column/cartridge overall.

According to another aspect, a method for separating an analyte from a solution is provided. The method comprises contacting the composite sorbent or the coated composite sorbent with the solution to form an analyte-bound composition; and separating the solution from the bound composition to provide a regenerated solution, wherein the amount of the analyte in the regenerated solution is less than the amount of the analyte in the solution. In an embodiment, the amount of the analyte in the regenerated solution can be about 10 to about 95% less, about 10 to about 90% less, about 15 to about 85% less, about 20 to about 80% less, about 25 to about 75% less, about 30 to about 70% less, about 35 to about 65% less, or about 40 to about 60% less than the amount of the analyte in the solution, based on the total weight of the regenerated solution or solution.

In an embodiment, the method for separating the analyte from the solution further includes contacting a solvent with the analyte-bound composition, wherein at least a portion of the analyte is removed from the analyte-bound composition and into the solvent. Suitable solvents include those capable of dissolving the analyte, preferably without disturbing the composite sorbent or the coated composite sorbent. For example, the solvent can be a ketone solvent such as methyl isobutyl ketone, 1-methyl-2-pyrrolidinone (NMP), cyclohexanone, acetone, or the like; an alcohol solvent such as isopropyl alcohol, butanol, or the like; an amide solvent such as dimethylacetamide, dimethylformamide (DMF), or the like; a nitrile solvent such as acetonitrile, benzonitrile, or the like; or a mixture of solvents. In an embodiment, the one or more steps of the method of separating the analyte from the solution can be performed in the device as described herein.

The method and device for separating the analyte from the solution can be used for hemodialysis. The composite sorbent or the coated composite sorbent can be suitable for removing substances from biofluids, blood, and blood plasma and from hemodialysis and peritoneal fluid. The composite sorbent material can for instance be used in a wearable artificial kidney, as a direct additive to a dialysate (a so-called sorption-additive) or any other fluid, from which the removal of specific substances, in particular toxic substances, is required. In addition to purifying blood, blood plasma, and dialysis fluid, the composite sorption materials can also be used for purification of other biofluids such as fluids extracted from the body that are subject for detailed analysis such as DNA profiling including PCR magnification.

According to another aspect, a hemodialysis or hemofiltration system is described for using the methods or devices herein. In an embodiment, the solution is a dialysate and the analyte is urea, creatinine, uremic acid, or a combination comprising at least one of the foregoing. In an embodiment, the system further includes a first analyte sensor to determine the concentration of the analyte in the dialysate and a second analyte sensor to determine the concentration of the analyte in the regenerated dialysate.

Hemodialysis, as used herein, includes all forms of therapies to remove waste, toxins, and excess water from a patient. The hemo therapies, such as hemodialysis, hemofiltration and hemodiafiltration, include both intermittent therapies and continuous therapies used for continuous renal replacement therapy (CRRT). The continuous therapies include, for example, slow continuous ultrafiltration (SCUF), continuous venovenous hemofiltration (CVVH), continuous venovenous hemodialysis (CVVHD), continuous venovenous hemodiafiltration (CVVHDF), continuous arteriovenous hemofiltration (CAVH), continuous arteriovenous hemodialysis (CAVHD), continuous arteriovenous hemodiafiltration (CAVHDF), continuous ultrafiltration periodic intermittent hemodialysis, or the like. The present invention can also be used during peritoneal dialysis including, for example, continuous ambulatory peritoneal dialysis, automated peritoneal dialysis, continuous flow peritoneal dialysis, or the like.

Further, although composite sorbent compositions, coated composite sorbent compositions, and articles including the same can be utilized in methods providing a dialysis therapy for patients having chronic kidney failure or disease, it should be appreciated that they can be used for acute dialysis needs, for example, in an emergency room setting. For example, the composite sorbent compositions, coated composite sorbent compositions, and articles including the same can be used to remove toxins from biological fluids, including toxins such as dimethylamine, ethylamine, monomethylamine, noradrenalin, trimethylamine, trimethylamine-n-oxide, dihydroxyphenylalanine, putrescine, spermidine, spermine, anthranilic acid, cysteine, carboxymethyllysine, hippuric acid, homocysteine, alpha-keto-d-guanidinovaleric acid, argininic acid, asymmetric dimethylarginine, symmetric dimethylarginine, guanidine, guanidinoacetic acid, guanidino succinic acid, methylguanidine, 4-pyridone-3-carboxamide-1-b-dribonucleoside, nicotinamide, N-methyl-2-pyridone-5-carboxamide, N-methyl-4-pyridone-3-carboxamide, 8-hydroxy-2′-deoxyguanosine, hypoxanthine, neopterin, indican, indole-3-acetic acid, indoxyl sulfate, indoxyl-b-d-glucoronide, or kynurenic acid. In addition, when a layer of the composite sorbent compositions, coated composite sorbent compositions, and articles including the same are combined with a secondary sorbent in a device, the resulting devices can be suitable for removing additional components from fluids such as alpha1-acid glycoprotein, alpha1-microglobulin, beta-trace protein, beta 2-microglobulin, adiponectin, angiogenin, calcitonin, complement factor D, cystatin C, fibroblast growth factor-23, glutathione, IGF-1, interleukin-6, Interleukin-8, Interleukin-10, leptin, myoglobin, osteocalcin, prolactin, resistin, retinol binding protein, or TNF-alpha.

It should be appreciated that the composite sorbent compositions, coated composite sorbent compositions, and articles including the same can be effectively utilized with a variety of different applications, physiologic and non-physiologic, in addition to hemodialysis. The composite sorbent compositions, coated composite sorbent compositions, articles including the same can be suitable for applications such as industrial processes, environmental protection processes, and for purification of organic compounds and pharmaceutical ingredients. For example, the composite sorbent compositions, coated composite sorbent compositions, and articles including the same can be used for the removal of nitrogen containing inorganic and organic compounds including ammonia, (substituted) hydroxylamines, (substituted) hydrazines, hydrazines and hydrazides, (substituted) acyclic, cyclic and heterocyclic guanidines, (substituted) acyclic, cyclic and heterocyclic alkyl- and aryl-ureas, (substituted) acyclic, cyclic and heterocyclic alkyl- and aryl-thioureas, aliphatic, aromatic, alkylaromatic, acyclic, cyclic and heterocyclic amines. It should be appreciated that the composite sorbent compositions, coated composite sorbent compositions, and articles including the same can chemically bind the constituents of any suitable fluid existing in liquid phase, gaseous phase, mixed liquid and gaseous phase, supercritical systems, or the like.

This disclosure is further illustrated by the following examples, which are non-limiting.

### EXAMPLES

The following materials were used (Table 1).

TABLE 1

| Component | Description | CAS# | Source |
|---|---|---|---|
| Alloxan monohydrate | 2,4,5,6(1H,3H)-Pyrimidinetetrone | 2244-11-3 | Aldrich |
| DOWEX OPTIPORE V-493 | Poly(styrene-co-divinylbenzene), polystyrene | 69011-14-9 | Dow Chemical |
| | crosslinked with divinylbenzene | | |
| DOWEX OPTIPORE V-503 | Poly(styrene-co-divinylbenzene), polystyrene | 69011-14-9 | Dow Chemical |
| | crosslinked with divinylbenzene | | |
| AMBERLITE XAD7HP | Poly(aliphatic-acrylate) polymer | N/A | Aldrich |
| AMBERLITE XAD4 | Poly(styrene-co-divinylbenzene), polystyrene | 9003-69-4 | Aldrich |
| | crosslinked with divinylbenzene | | |
| Poly-VMK | Poly(vinyl methyl ketone) | 25038-87-3 | Aldrich |
| Poly-HEMA | Poly(2-hydroxyethyl methacrylate) | 25249-16-5 | Sigma |
| Poly(vinyl alcohol) | Poly(vinyl alcohol) | 9002-89-5 | Aldrich |
| Poly(acrylamide) | Poly(acrylamide) | 9003-05-8 | Aldrich |
| Potassium acetate | Potassium acetate | 127-08-2 | Aldrich |
| Glutaraldehyde solution | Glutaraldehyde solution | 111-30-8 | Aldrich |
| HCl | Concentrated hydrochloric acid (37%, reagent grade) | 7647-01-0 | Aldrich |
| DI water | Deionized water having a purity of at least 99.9% | 7732-18-5 | Aldrich |
| Ninhydrin | 2,2-Dihydroxy-1,3-indanedione | 485-47-2 | Aldrich |
| Macroporous | Macroporous polymer of technical 60%-divinyl benzene | | Example XII of |
| poly(divinyl benzene) | | | U.S. Pat. No. |
| | | | 4,897,200 |
| Urea | Carbonyldiamine | 57-13-6 | Aldrich |
| Glacial acetic acid | Acetic acid (99.8%, glacial) | 64-19-7 | Aldrich |
| DMABA | Dimethylaminobenzaldehyde | 100-10-7 | Aldrich |

All reagents were used as received unless noted otherwise.

### Analytical Methods.

Specific surface area, pore volume, average pore diameter, and pore size distribution were determined by nitrogen adsorption using a Quantachrome-Autosorb 6iSA instrument according to ASTM UOP964-11.

Elemental analysis (% CHN) was performed according to ASTM D5291 using a 2400 Series II CHNS/O Elemental Analyzer from Perkin Elmer.

Ultraviolet-visible (UV/vis) absorbance spectroscopy was performed using a Lambda 35 spectrophotometer from Perkin Elmer.

Fourier transform infrared (FTIR) spectroscopy was performed on Nicolet 8700 FTIR spectrometer with MTEC M300 Photoacoustic Accessory using 200 scans at a resolution of 4 cm⁻¹.

### General Preparation of a Two-Component Composite Sorbent.

A reaction vial equipped with a magnetic stirring bar was charged with a 1.2 molar (M) solution of an extractant in deionized (DI) water and a polymeric adsorbent (4 g). The reaction vial was capped and the contents were stirred for 8 hours at 25° C. The resulting composite sorbent was filtered, rinsed with DI water (10 mL), and dried at 70° C. under vacuum for 12 hours. The obtained composite sorbent was characterized by FTIR and had peaks at ˜3350 inverse centimeters (cm⁻¹) and at 1750 cm⁻¹ attributable to NH vibrations and carbonyl stretching, respectively. The amount of extractant adsorbed by the adsorbent polymer was calculated based on the sample weight gain and analysis by nitrogen content according to ASTM D5291.

### Example 1

A 40 mL reaction vial equipped with a magnetic stirring bar was charged with alloxan monohydrate (2.14 g, 13.4 millimoles (mmol)), DOWEX OPTIPORE V-493 (4 g), and DI water (10 mL). The reaction vial was capped and the contents were stirred for 8 hours at 25° C. The resulting product was filtered, rinsed with DI water (10 mL), and dried in a vacuum oven at 70° C. for 12 hours resulting in 4.88 g of the composite sorbent (0.88 g weight gain, 18 wt % extractant content) with a nitrogen content of 3.33 weight percent (wt %).

### Example 2

A 40 mL reaction vial equipped with a magnetic stirring bar was charged with alloxan monohydrate (1.6 g, 10 mmol), DOWEX OPTIPORE V-503 (4 g), and DI water (10 mL). The reaction vial was capped and the contents were stirred for 8 hours at 25° C. The resulting product was filtered, rinsed with DI water (10 mL), and dried in a vacuum oven at 70° C. for 12 hours resulting in 5.04 g of the composite sorbent (1.04 g weight gain, 21 wt % of the extractant) with a nitrogen content of 3.71 wt %.

### Example 3

A 40 mL reaction vial equipped with a magnetic stirring bar was charged with alloxan monohydrate (2.14 g, 13.4 mmol), AMBERLITE XAD7HP (4 g), and DI water (10 mL). The reaction vial was capped and the contents were stirred for 8 hours at 25° C. The resulting product was filtered, rinsed with DI water (10 mL), and dried in a vacuum oven at 70° C. for 12 hours resulting in 5.07 g of the composite sorbent (1.07 g weight gain, 21 wt % of the extractant) with a nitrogen content of 3.82 wt %.

### Example 4

A 40 mL reaction vial equipped with a magnetic stirring bar was charged with alloxan monohydrate (2.14 g, 13.4 mmol), AMBERLITE XAD4 (4 g), and DI water (10 mL). The reaction vial was capped and the contents were stirred for 8 hours at 25° C. The resulting product was filtered, rinsed with DI water (10 mL), and dried in a vacuum oven at 70° C. for 12 hours resulting in 4.73 g of the composite sorbent (0.73 g weight gain, 15 wt % of the extractant) with a nitrogen content of 2.61 wt %.

### Comparative Example 1

A 40 mL reaction vial equipped with a magnetic stirring bar was charged with AMBERLITE XAD4 (4 g) and DI water (10 mL). The reaction vial was capped and the contents were stirred for 8 hours at 25° C. The resulting product was filtered, rinsed with DI water (10 mL), and dried in a vacuum oven at 70° C. for 12 hours resulting in 4 g of the polymeric adsorbent (no weight gain, no extractant content) with a nitrogen content of 0.2 wt %.

### Comparative Example 2

According to Example XII of U.S. Pat. No. 4,897,200 a composite sorbent was prepared using ninhydrin and a polymer adsorbent. 80 g of a macroporous polymer of technical 60%-divinyl benzene having a surface area of 740 m²/g and an apparent density of 0.50 g/ml were suspended in DI water (500 mL), followed by adding ninhydrin (20 g, 112 mmol) at a temperature of 50° C. After 6 hours' stirring the mixture was cooled with stirring and the product was washed with water and dried. The resulting composite sorbent contained 20 wt % of the extractant.

### General Procedure for Measuring Urea Sorption.

All urea measurements were performed in an aqueous buffer solution prepared according to the following procedure.

In a 1,000 mL volumetric flask, NaCl (5.80 g), CaCl2 (333 mg), KCl (149 mg), MgCl2 (71 mg), dextrose (2.00 g), NaHCO₃ (2.77 g), and urea (2.00 g) were combined in DI water (approximately 500 mL). Glacial acetic acid (50 mL) was slowly added to the flask and gently swirled until all of the salts are dissolved. The flask was filled to the volumetric mark with DI water resulting in solution A.

A buffer solution, solution B, was prepared in a similar manner without urea.

To solution A (100 mL) was added to the sorbent sample (1 g) and the vial was shaken for 24 hours. A 1.7 mL aliquot of the resulting solution was diluted with buffer solution B (2.5 mL). To this was added a dimethylaminobenzaldehyde (DMABA) solution (0.8 mL) that was prepared from DMABA (0.8 g), concentrated hydrochloric acid (2 mL), and the buffer solution B (18 mL). The UV/Vis absorbance of the resulting solution was measured at 420 nanometers (nm) to calculate the urea content based on a calibration curve. The urea content of the solution is reported as the urea sorption in grams per kilogram (g/kg), and represents the grams of urea that is adsorbed per kilogram of composite adsorbent.

The sorption results for urea using the composite sorbents of Examples 1 to 4, the polymeric adsorbent of Comparative Example 1, and the coated polymer of Comparative Example 2 are presented in Table 2.

TABLE 2

| | | Specific | | | Sample | | | |
|---|---|---|---|---|---|---|---|---|
| | | Surface | Pore | Pore | weight | Extractant | Nitrogen | Urea |
| | Polymeric | Area | Diameter | Volume | gain | loading | content^{†} | Sorption |
| Ex. | Adsorbent | (m²/g) | (Å) | (mL/g) | (g) | (wt %) | (wt %) | (g/kg) |
| 1 | OPTIPORE V-493 | 1100 | 46 | 1.16 | 0.88 | 18 | 3.33 | 22.3 |
| 2 | OPTIPORE V-503 | 1100 | 34 | 0.94 | 1.04 | 21 | 3.71 | 25.4 |
| 3 | Amberlite XAD7HP | 450 | 90 | 0.5 | 1.07 | 21 | 3.82 | 26.1 |
| 4 | Amberlite XAD4 | 725 | 50 | 0.98 | 0.73 | 15 | 2.61 | 19.1 |
| C1 | Amberlite XAD4 | 725 | 50 | 0.98 | — | — | 0.2 | 0.0 |
| C2* | Macroporous | 740 | — | — | 20 | 20 | — | 24 |
| | poly(divinyl | | | | | | | |
| | benzene) ^{‡} | | | | | | | |

^{†}Nitrogen content as measured before sorption of urea
^{‡} Ninhydrin extractant
*Data obtained from U.S. Pat. No. 4,897,200

### Example 5

A three-component composite sorbent was prepared by precipitation of a water-insoluble, water-permeable polymer on the surface of the composite sorbent of Example 1. A 40 mL reaction vial equipped with a magnetic stirring bar was charged with poly-VMK (0.140 g), potassium acetate (98 mg, 0.001 mol, porogen), the composite sorbent of Example 1 (1.5 g), and NMP (10 mL). The reaction vial was capped and stirred at room temperature (25° C.) for 1 hour. The resulting product was filtered, washed with methanol (10 mL) and DI water (5×20 mL), and dried in a vacuum oven at 70° C. for 12 hours. 1.59 g of the coated composite sorbent was obtained with a urea sorption capacity of 19.4 g/kg.

### Example 6

A three-component composite sorbent was prepared with thermal cross-linking. A 40 mL reaction vial equipped with a magnetic stirring bar was charged with an aqueous solution of polyvinyl alcohol (25 mL, 2 g/dL) and the composite sorbent of Example 1 (1.5 g). The reaction vial was capped and shaken at room temperature (25° C.) for 1 hour. The resulting product was filtered, washed with DI water (10 mL) and methanol (20 mL), and dried in a vacuum oven at 70° C. for 12 hours. The resultant material was heated at 200° C. for 1 hour to yield 1.71 g of the coated composite sorbent having a urea sorption capacity of 19.4 g/kg.

### Example 7

A three-component composite sorbent was prepared with thermal crosslinking. A 40 mL reaction vial equipped with a magnetic stirring bar was charged with an aqueous solution of poly-HEMA (25 mL, 1 g/dL), polyacrylic acid (5 mL, 1 g/L), and the composite sorbent of Example 2 (1.5 g). The reaction vial was capped and stirred at room temperature (25° C.) for 1 hour. The resulting product was filtered, washed with DI water (10 mL) and methanol (20 mL), and dried in a vacuum oven at 70° C. for 12 hours. The material was then heated at 200° C. for 20 minutes to yield 1.66 g of the coated composite sorbent having a urea sorption capacity of 19.4 g/kg

### Example 8

A three-component composite sorbent was prepared with chemical catalytic crosslinking. A 40 mL reaction vial equipped with a magnetic stirring bar was charged with an aqueous solution of polyvinyl alcohol (25 mL, 2 g/dL), citric acid (1 mL, 5% aqueous solution), and the composite sorbent of Example 2 (1.5 g). The reaction vial was capped and shaken at room temperature (25° C.) for 1 hour. The resulting product was filtered, washed with DI water (10 mL) and methanol (20 mL), and dried in a vacuum oven at 70° C. for 12 hours. The material was then heated in an oven at 200° C. for 1 hour to yield 1.84 g of the coated composite sorbent having a urea sorption capacity of 20.7 g/kg.

### Example 9

A three-component composite sorbent was prepared with a crosslinking reagent. A 40 mL reaction vial equipped with a magnetic stirring bar was charged with an aqueous solution of polyacrylamide (25 mL, 1 g/dL) and the composite sorbent of Example 2 (1.2 g). The reaction vial was capped and shaken at room temperature (25° C.) for 1 hour. The resulting product was filtered, transferred to reaction vial, and then re-suspended in DI water (10 mL) as solid beads. To this was added glutaraldehyde (0.5 mL, 5% aqueous solution) and hydrochloric acid (1 mL, 1 M aqueous solution). The reaction vial was capped and shaken for 1 hour. The resulting beads were filtered, washed with DI water (10 mL) and methanol (20 mL), and dried in a vacuum oven at 70° C. for 12 hours. 1.44 g of the coated composite sorbent was obtained with a urea sorption capacity of 16.8 g/kg.

### Example 10

Polypropylene solid phase extraction (SPE) cartridges may be prepared, each with a composite sorbent of Examples 1 to 9. A layer of the composite sorbent of one of Examples 1 to 9 may be prepared and then an additional layer of a secondary adsorbent may be added as a secondary layer as described below. The secondary adsorbents are shown in Table 3.

TABLE 3

| | |
|---|---|
| A | Activated carbon |
| B | Nanocarbon sorbent |
| C | Modified silica |
| D | Silica |
| E | Supplementary polymer adsorbent |
| F | Polymer ion exchanger, cationic strong |
| G | Polymer ion exchanger, anionic strong |
| H | Polymer ion exchanger, cationic weak |
| I | Polymer ion exchanger, anionic weak |
| J | Inorganic sorbent/ion exchanger, zeolite |
| K | Inorganic sorbent, zirconium oxide |
| L | Inorganic sorbent, zirconium phosphate |
| M | Inorganic sorbent, nanoclay |

In the device, the composite sorbent layer can include 5 to 95 wt %, preferably 10 to 90 wt % of the total adsorbent weight. One or more of the secondary adsorbents (Table 3) can be included as a single layer or multiple layers that are downstream, upstream or both, of the composite sorbent layer along the length of the column (for example, a cartridge). The column diameter to length ratio can be 1:200 to 1:1, preferably 1:20 to 1:2. Each layer can have a thickness of 1 to 1,000 millimeters (mm). A packed column can be prepared by adding the composite sorbent and the one or more secondary adsorbents into the cartridge housing using standard techniques, e.g. loading of dry powder or/and beads, or adding a suspension in the appropriate solvent or solvent mixtures. The chromatographic column can then be ultrasonicated or pressure can be applied to the packing material to improve the uniformity of each layer and column/cartridge overall.

This disclosure further encompasses the following embodiments.

Embodiment 1. A composite sorbent composition comprising a polymeric adsorbent; and an extractant having the formula

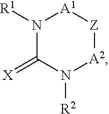

or hydrate thereof, wherein X is O or S, A¹ and A² are each independently —C(O)— or —C(R′)(R″)— wherein R′, and R″ are each independently hydrogen, halogen, hydroxyl, cyano, nitro, amino, —CHO, —COOH, C₁₋₁₂ alkyl, C₁₋₄ alkoxy, C₁₋₄ alkylamino, C₁₋₂ haloalkyl, C₁₋₂ haloalkoxy, C₁₋₁₂ cycloalkyl, C₆₋₁₂ aryl, C₇₋₁₃ arylalkyl, C₃₋₁₂ heteroaryl, C₁₋₁₂ heteroalkyl, or C₄₋₁₂ heteroarylalkyl, Z is a covalent bond, —S—, —O—, —SO₂—, —SO—, —P(R)(═O)—, —NR—, —C(O)—, —C(O)NH—, —C(═N—R)—, or —C(R′)(R″)— wherein R, R′, and R″ are each independently hydrogen, halogen, hydroxyl, cyano, nitro, amino, —CHO, —COOH, —C(O)NH₂, C₁₋₁₂ alkyl, C₁₋₁₂ alkoxy, C₁₋₁₂ alkylamino, C₁₋₄ haloalkyl, C₁₋₄ haloalkoxy, C₄₋₁₂ cycloalkyl, C₆₋₁₂ aryl, C₇₋₁₃ arylalkyl, C₃₋₁₂ heterocycloalkyl, C₃₋₁₂ heteroaryl, C₁₋₁₂ heteroalkyl, or C₄₋₁₂ heteroarylalkyl, and R¹ and R² are each independently hydrogen, halogen, hydroxyl, cyano, nitro, amino, or a substituted or unsubstituted monovalent C₁₋₄₀ hydrocarbon.

Embodiment 2. The composition of embodiment 1, wherein the extractant has the formula

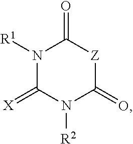

or a hydrate thereof, wherein X is O or S, Z is a covalent bond, —S—, —O—, —SO₂—, —SO—, —P(R)(═O)—, —NR—, —C(O)—, —C(O)NH—, —C(═N—R)—, or —C(R′)(R″)— wherein R, R′, and R″ are each independently hydrogen, halogen, hydroxyl, cyano, nitro, amino, —CHO, —COOH, —C(O)NH₂, C₁₋₁₂ alkyl, C₁₋₁₂ alkoxy, C₁₋₁₂ alkylamino, C₁₋₄ haloalkyl, C₁₋₄ haloalkoxy, C₄₋₁₂ cycloalkyl, C₆₋₁₂ aryl, C₇₋₁₂ arylalkyl, C₃₋₁₂ heterocycloalkyl, C₃₋₁₂ heteroaryl, C₁₋₁₂ heteroalkyl, or C₄₋₁₂ heteroarylalkyl, and R¹ and R² are each independently hydrogen, C₁₋₁₂ alkyl, C₂₋₁₂ alkenyl, C₂₋₁₂ alkynyl, C₄₋₁₂ cycloalkyl, C₆₋₁₂ aryl, C₇₋₁₂ arylalkyl, C₃₋₁₂ heterocycloalkyl, C₃₋₁₂ heteroaryl, C₁₋₁₂ heteroalkyl, or C₄₋₁₂ heteroarylalkyl, each of which R¹ and R² is unsubstituted or substituted with one or more of halogen, hydroxyl, cyano, nitro, amino, —CHO, —COOH, —C(O)NH₂, C₁₋₁₂ alkyl, C₂₋₁₂ alkenyl, C₂₋₁₂ alkynyl, C₁₋₁₂ alkoxy, C₂₋₁₂ alkanoyl, mono- or di(Ci-12 alkylamino) C₀₋₈ alkyl, (C₁₋₁₂ alkyl)carboxamide, (C₁₋₁₂ alkyl) ester, C₁₋₁₂ heteroalkyl, C₁₋₄ haloalkyl, or C₁₋₄ haloalkoxy.

Embodiment 3. The composition of embodiment 1 or embodiment 2, wherein Z is —C(R′)(R″)— wherein R′ and R″ are each independently hydrogen, halogen, hydroxyl, amino, C₁₋₄ alkyl, C₁₋₄ alkoxy, C₁₋₄ alkylamino, C₁₋₆ alkylthio, C₁₋₆ alkylsulfonyl, trifluoromethyl, difluoromethyl, or trifluoromethoxy.

Embodiment 4. The composition of any one or more of embodiments 1 to 3, wherein X is O and Z is —C(O)—.

Embodiment 5. The composition of any one or more of embodiments 1 to 4, wherein R¹ and R² are the same, and are hydrogen or C₁₋₆ alkyl.

Embodiment 6. The composition of any one or more of embodiments 1 to 5, wherein R¹ and R² are each hydrogen, X is O, and Z is —C(O)— or —C(R′)(R″)— wherein R′ and R″ are each hydroxyl.

Embodiment 7. The composition of any one or more of embodiments 1 to 6, wherein the polymeric adsorbent is derived from a monomer comprising a vinyl C₆₋₁₂ aryl, a divinyl C₆₋₁₂ aryl, a trivinyl C₆₋₁₂ aryl, a (C₁₋₈ alkyl) (meth)acrylate, an alkylenediol di(meth)acrylate, an alkylenetriol tri(meth)acrylate, a polyester di(meth)acrylate, a (meth)acrylamide, a bis(meth)acrylamide, or a combination comprising at least one of the foregoing.

Embodiment 8. The composition of any one or more of embodiments 1 to 7, wherein the polymeric adsorbent comprises poly(styrene-divinylbenzene), sulfonated poly(styrene-divinylbenzene), poly(ethylvinylbenzene-divinylbenzene), poly(amide-divinylbenzene), poly(N-vinylpyrrolidone-divinylbenzene), poly((meth)acrylate-divinylbenzene), poly((meth)acrylonitrile-divinylbenzene), poly(2-hydroxyethyl (meth)acrylate-ethylstyrene-divinylbenzene), poly(cyanomethylstyrene-divinylbenzene), poly(4-vinylpyridine-divinylbenzene), poly(N-vinylimidazole-divinylbenzene), poly(4-vinylimidazole-divinylbenzene), poly(1-vinyl-2-pyrrolidone-divinylbenzene), poly(para-vinylbenzylchloride-divinylbenzene), poly(meta/para-vinylbenzylchloride-divinylbenzene), poly(2-hydroxyethyl (meth)acrylate-vinylbenzylchloride-divinylbenzene), a poly((C₁₋₈ alkyl) (meth)acrylate), or a combination comprising at least one of the foregoing.

Embodiment 9. The composition of any one or more of embodiments 1 to 8, wherein the polymeric adsorbent has an average pore diameter of about 10 to about 1,400 Angstroms, preferably about 12 to about 1,200 Angstroms, more preferably about 14 to about 1,000 Angstroms; a pore volume of about 0.1 to about 2.25 milliliters per gram, preferably about 0.2 to about 2.0 milliliters per gram, more preferably about 0.3 to about 1.85 milliliters per gram; and a specific surface area of about 50 to about 2,500 square meters per gram, preferably about 100 to about 1,800 square meters per gram, more preferably about 150 to about 1,200 square meters per gram.

Embodiment 10. The composition of any one or more of embodiments 1 to 9, further comprising: a precipitated water-insoluble polymer on the surface of the composition, wherein the water-insoluble polymer is derived from a monovinyl aromatic monomer, a monovinylic monomer, a C₆₋₁₂ aryl sulfone, or a combination comprising at least one of the foregoing, preferably styrene, alpha-ethyl styrene, vinyl naphthalene, vinylbenzyl alcohol, acrylonitrile, methacrylic acid, vinyl chloride, vinylidene chloride, vinyl formamide, a vinyl C₁₋₆ alkyl ketone, a C₁₋₆ alkyl vinyl ether, a vinyl C₃₋₈ carboxylate, a C₁₋₆ alkyl (meth)acrylate, a C₆₋₁₂ aryl sulfone, or a combination comprising at least one of the foregoing; or a cross-linked product of a precipitated water-soluble polymer on the surface of the composition, wherein the precipitated water-soluble polymer comprises a poly(vinyl alcohol), a poly((C₁₋₆ alkyl) hydroxy (meth)acrylate, a hydroxy (C₁₋₆ alkyl) cellulose, starch, dextrin, an alkali or ammonium acid salt of a carboxy(C₁₋₃ alkyl) cellulose ether, a poly(di(C₁₋₆ alkyl)aminoethyl (meth)acrylate), poly(N-vinylpyrrolidone), an alkali or ammonium salt of poly(meth)acrylic acid, a poly(meth)acrylamide or a partially hydrolyzed derivative thereof, a poly(N-(C₁₋₆ alkyl)(meth)acrylamide), a poly(N,N-di(C₁₋₆ alkyl)(meth)acrylamide, 2-(meth)acrylamido-2-methylpropane sulfonic acid or an alkali salt thereof, or a combination comprising at least one of the foregoing.

Embodiment 11. A method for the manufacture of the composition of any one or more of embodiments 1 to 9, the method comprising contacting the extractant and the polymeric adsorbent in a solvent under conditions effective to provide the composition.

Embodiment 12. The method of embodiment 11, further comprising precipitating a water-insoluble polymer on a surface of the composition to provide the composition of embodiment 10.

Embodiment 13. The method of embodiment 11, further comprising: precipitating a water-soluble polymer on a surface of the composition; and cross-linking the water-soluble polymer on the surface to provide the composition of embodiment 10.

Embodiment 14. A device comprising the composition of any one or more of embodiments 1 to 10, or made by any one or more of the methods of embodiments 11 to 13.

Embodiment 15. The device of embodiment 14, further comprising at least one secondary adsorbent, wherein the at least one secondary adsorbent is activated carbon, silica, modified silica, a second polymeric adsorbent, or a combination comprising at least one of the foregoing.

Embodiment 16. A method for separating an analyte from a solution, the method comprising: contacting the composition of any one or more of embodiments 1 to 10 or made by any one or more of the methods of embodiments 11 to 13 with the solution to form an analyte-bound composition; and separating the solution from the analyte-bound composition to provide a regenerated solution, wherein the amount of the analyte in the regenerated solution is less than the amount of the analyte in the solution.

Embodiment 17. The method of embodiment 16, further comprising contacting a solvent with the analyte-bound composition, wherein at least a portion of the analyte is removed from the analyte-bound composition and into the solvent.

Embodiment 18. The method of embodiment 16 or 17, wherein one or more of the steps are performed in the device of embodiments 14 or 15.

Embodiment 19. A hemodialysis or hemofiltration system for using the methods of any one or more of embodiments 16 to 18, wherein the solution is a dialysate and wherein the analyte is urea, creatinine, uremic acid, or a combination comprising at least one of the foregoing.

Embodiment 20. The system of embodiment 19, further comprising a first analyte sensor to determine the concentration of the analyte in the dialysate and a second analyte sensor to determine the concentration of the analyte in the regenerated dialysate.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt %, or, more specifically, 5 to 20 wt %”, is inclusive of the endpoints and all intermediate values of the ranges of “5 to 25 wt %,” etc.). “Combinations” is inclusive of blends, mixtures, alloys, reaction products, or the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group. The term “alkyl” means a branched or straight chain, unsaturated aliphatic hydrocarbon group, e.g., methyl, ethyl, n-propyl, i-propyl, n-butyl, s-butyl, t-butyl, n-pentyl, s-pentyl, and n- and s-hexyl. “Alkenyl” means a straight or branched chain, monovalent hydrocarbon group having at least one carbon-carbon double bond (e.g., ethenyl (—HC═CH₂)). “Alkylene” means a straight or branched chain, saturated, divalent aliphatic hydrocarbon group (e.g., methylene (—CH2—) or, propylene (—(CH₂)₃—)). “Cycloalkyl” means a monovalent group having one or more rings and one or more carbon-carbon double bonds in the ring, wherein all ring members are carbon (e.g., cyclopentyl and cyclohexyl). “Carbocyclic groups” include cycloalkyl groups, such as cyclopropyl and cyclohexyl; cycloalkenyl groups, such as cyclohexenyl, bridged cycloalkyl groups; and aryl groups, such as phenyl.

“Halo” or “halogen” means fluoro, chloro, bromo, or iodo. “Alkoxy” mean s an alkyl group that is linked via an oxygen (i.e., alkyl-O—), for example methoxy, ethoxy, and sec-butyloxy groups. “Haloalkoxy” means a haloalkyl group as defined above attached through an oxygen bridge (oxygen of an alcohol radical).“Aryl” means an aromatic hydrocarbon group containing the specified number of carbon atoms, such as phenyl, tropone, indanyl, or naphthyl. “Arylene” means a divalent aryl group. “Arylalkyl” means an alkyl group substituted with an aryl group (e.g., benzyl). “Aryloxy” means an aryl group attached to the group it substitutes through an oxygen bridge.

“Alkylcarboxamide” means a group of the formula —(C═O)Nalkyl₁alkyl₂, where the alkyl₁ and alkyl₂ groups are independently the same or different alkyl groups, attached through a carboxamide linkage. The carboxamide linkage can be in either orientation, e.g., —NH(C═O)— or (C═O)NH—. “Alkyl ester” means an alkyl group attached through an ester linkage. The ester linkage may be in either orientation, e.g., a group of the formula —O(C═O)alkyl or a group of the formula —(C═O)—)Oalkyl. “Alkanoyl” means an alkyl group attached through a keto (—(C═O)—) bridge. Alkanoyl groups have the indicated number of carbon atoms, with the carbon of the keto group being included in the numbered carbon atoms. “Alkylamino” includes both mono- and di-alkylamino groups, and means a secondary or tertiary alkyl amino group, wherein the alkyl groups have the indicated number of carbon atoms. The point of attachment of the alkylamino group is on the nitrogen.

The prefix “hetero” means that the compound or group includes at least one ring member that is a heteroatom (e.g., 1, 2, or 3 heteroatom(s)), wherein the heteroatom(s) is each independently N, O, S, Si, or P. “Heteroaryl” means a monovalent carbocyclic ring group that includes one or more aromatic rings, in which at least one ring member (e.g., one, two or three ring members) is a heteroatom. “Heteroarylalkyl” means a heteroaryl group linked via an alkyl moiety. “Heteroaryloxy” means a heteroaryl group is attached to the group it substitutes through an oxygen bridge. “Heterocycloalkyl” means a saturated cyclic group having the indicated number of ring atoms containing from 1 to about 3 heteroatoms (e.g., N, O, or S), with remaining ring atoms being carbon. Examples of heterocycloalkyl groups include tetrahydrofuranyl and pyrrolidinyl groups.

Unless indicated otherwise, “substituted” means that the compound or group is substituted with at least one (e.g., 1, 2, 3, or 4) substituents that can each independently be a C₁₋₉ alkoxy, a C₁₋₉ haloalkoxy, a nitro (—NO₂), a cyano (—CN), a C₁₋₆ alkyl sulfonyl (—S(═O)₂-alkyl), a C₆₋₁₂ aryl sulfonyl (—S(═O)₂-aryl), a thiol (—SH), a thiocyano (—SCN), a tosyl (CH₃C₆H₄SO₂—), a C₃₋₁₂ cycloalkyl, a C₂₋₁₂ alkenyl, a C₅₋₁₂ cycloalkenyl, a C₆₋₁₂ aryl, a C₇₋₁₃ arylalkylene, a C₄₋₁₂ heterocycloalkyl, and a C₃₋₁₂ heteroaryl instead of hydrogen, provided that the substituted atom's normal valence is not exceeded. The number of carbon atoms indicated in a group is exclusive of any substituents. For example —CH₂CH₂CN is a C₂ alkyl group substituted with a cyano.

“Acryl” used herein includes both acrylics and acrylates. “Methacryl” used herein includes both (meth)acrylics and (meth)acrylates. The prefix “(meth)” includes both the acryl and methacryl forms.

“Vinyl” group includes any group having terminal unsaturation (—CH_{2═}CH₂), including acrylate groups (—OC(O)CH═CH₂) and methacrylate groups (—OC(O)(CH₃)═CH₂).

As used herein, “adsorbent” means a condensed phase at the surface of which adsorption can occur.

As used herein, “extractant” means an active component that aids in the transfer of a solute/analyte from one phase to another.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A composite sorbent composition comprising:
a polymeric adsorbent; and
an extractant having the formula 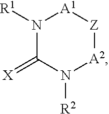
or a hydrate thereof,
wherein
X is O or S,
A¹ and A² are each independently —C(O)— or —C(R′)(R″)— wherein R′, and R″ are each independently hydrogen, halogen, hydroxyl, cyano, nitro, amino, —CHO, —COOH, C₁₋₁₂ alkyl, C₁₋₄ alkoxy, C₁₋₄ alkylamino, C₁₋₂ haloalkyl, C₁₋₂ haloalkoxy, C₁₋₁₂ cycloalkyl, C₆₋₁₂ aryl, C₇₋₁₃ arylalkyl, C₃₋₁₂ heteroaryl, C₁₋₁₂ heteroalkyl, or C₄₋₁₂ heteroarylalkyl,
Z is a covalent bond, —S—, —O—, —SO₂—, —SO—, —P(R)(═O)—, —NR—, —C(O)—, —C(O)NH—, —C(═N—R)—, or —C(R′)(R″)— wherein R, R′, and R″ are each independently hydrogen, halogen, hydroxyl, cyano, nitro, amino, —CHO, —COOH, —C(O)NH₂, C₁₋₁₂ alkyl, C₁₋₁₂ alkoxy, C₁₋₁₂ alkylamino, C₁₋₄ haloalkyl, C₁₋₄ haloalkoxy, C₄₋₁₂ cycloalkyl, C₆₋₁₂ aryl, C₇₋₁₃ arylalkyl, C₃₋₁₂ heterocycloalkyl, C₃₋₁₂ heteroaryl, C₁₋₁₂ heteroalkyl, or C₄₋₁₂ heteroarylalkyl, and
R¹ and R² are each independently hydrogen, halogen, hydroxyl, cyano, nitro, amino, or a substituted or unsubstituted monovalent C₁₋₄₀ hydrocarbon.

2. The composition of claim 1, wherein the extractant has the formula 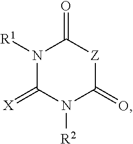
or a hydrate thereof, wherein
X is O or S,
Z is a covalent bond, —S—, —O—, —SO₂—, —SO—, —P(R)(═O)—, —NR—, —C(O)—, —C(O)NH—, —C(═N—R)—, or —C(R′)(R″)— wherein R, R′, and R″ are each independently hydrogen, halogen, hydroxyl, cyano, nitro, amino, —CHO, —COOH, —C(O)NH₂, C₁₋₁₂ alkyl, C₁₋₁₂ alkoxy, C₁₋₁₂ alkylamino, C₁₋₄ haloalkyl, C₁₋₄ haloalkoxy, C₄₋₁₂ cycloalkyl, C₆₋₁₂ aryl, C₇₋₁₂ arylalkyl, C₃₋₁₂ heterocycloalkyl, C₃₋₁₂ heteroaryl, C₁₋₁₂ heteroalkyl, or C₄₋₁₂ heteroarylalkyl, and
R¹ and R² are each independently hydrogen, C₁₋₁₂ alkyl, C₂₋₁₂ alkenyl, C₂₋₁₂ alkynyl, C₄₋₁₂ cycloalkyl, C₆₋₁₂ aryl, C₇₋₁₂ arylalkyl, C₃₋₁₂ heterocycloalkyl, C₃₋₁₂ heteroaryl, C₁₋₁₂ heteroalkyl, or C₄₋₁₂ heteroarylalkyl, each of which R¹ and R² is unsubstituted or substituted with one or more of halogen, hydroxyl, cyano, nitro, amino, —CHO, —COOH, —C(O)NH₂, C₁₋₁₂ alkyl, C₂₋₁₂ alkenyl, C₂₋₁₂ alkynyl, C₁₋₁₂ alkoxy, C₂₋₁₂ alkanoyl, mono- or di(C₁₋₁₂ alkylamino) C₀₋₈ alkyl, (C₁₋₁₂ alkyl)carboxamide, (C₁₋₁₂ alkyl) ester, C₁₋₁₂ heteroalkyl, C₁₋₄ haloalkyl, or C₁₋₄ haloalkoxy.

3. The composition of claim 1, wherein Z is —C(R′)(R″)—wherein R′ and R″ are each independently hydrogen, halogen, hydroxyl, amino, C₁₋₄ alkyl, C₁₋₄ alkoxy, C₁₋₄ alkylamino, C₁₋₆ alkylthio, C₁₋₆ alkylsulfonyl, trifluoromethyl, difluoromethyl, or trifluoromethoxy.

4. The composition of claim 1, wherein X is O and Z is —C(O)—.

5. The composition of claim 1, wherein R¹ and R² are the same, and are hydrogen or C₁₋₆ alkyl.

6. The composition of claim 1, wherein R¹ and R² are each hydrogen, X is O, and Z is —C(O)— or —C(R′)(R−)— wherein R′ and R″ are each hydroxyl.

7. The composition of claim 1, wherein the polymeric adsorbent is derived from a monomer that is a vinyl C₆₋₁₂ aryl, a divinyl C₆₋₁₂ aryl, a trivinyl C₆₋₁₂ aryl, a (C₁₋₈ alkyl) (meth)acrylate, an alkylenediol di(meth)acrylate, an alkylenetriol tri(meth)acrylate, a polyester di(meth)acrylate, a (meth)acrylamide, a bis(meth)acrylamide, or a combination thereof.

8. The composition of claim 1, wherein the polymeric adsorbent is poly(styrene-divinylbenzene), sulfonated poly(styrene-divinylbenzene), poly(ethylvinylbenzene-divinylbenzene), poly(amide-divinylbenzene), poly(N-vinylpyrrolidone-divinylbenzene), poly((meth)acrylate-divinylbenzene), poly((meth)acrylonitrile-divinylbenzene), poly(2-hydroxyethyl (meth)acrylate-ethylstyrene-divinylbenzene), poly(cyanomethylstyrene-divinylbenzene), poly(4-vinylpyridine-divinylbenzene), poly(N-vinylimidazole-divinylbenzene), poly(4-vinylimidazole-divinylbenzene), poly(1-vinyl-2-pyrrolidone-divinylbenzene), poly(para-vinylbenzylchloride-divinylbenzene), poly(meta/para-vinylbenzylchloride-divinylbenzene), poly(2-hydroxyethyl (meth)acrylate-vinylbenzylchloride-divinylbenzene), a poly((C₁₋₈ alkyl) (meth)acrylate), or a combination thereof.

9. The composition of claim 1, wherein the polymeric adsorbent has
an average pore diameter of 10 to 1,400 Angstrom;
a pore volume of 0.1 to 2.25 milliliters per gram; and
a specific surface area of 50 to 2,500 square meters per gram.

10. The composition of claim 1, further comprising:
a precipitated water-insoluble polymer on the surface of the composition, wherein the water-insoluble polymer is derived from a monovinyl aromatic monomer, a monovinylic monomer, a C₆₋₁₂ aryl sulfone, or a combination thereof; or
a cross-linked product of a precipitated water-soluble polymer on the surface of the composition, wherein the precipitated water-soluble polymer is a poly(vinyl alcohol), a poly((C₁₋₆ alkyl) hydroxy (meth)acrylate, a hydroxy (C₁₋₆ alkyl) cellulose, starch, dextrin, an alkali or ammonium acid salt of a carboxy(C₁₋₃ alkyl) cellulose ether, a poly(di(C₁₋₆ alkyl)aminoethyl (meth)acrylate), poly(N-vinylpyrrolidone), an alkali or ammonium salt of poly(meth)acrylic acid, a poly(meth)acrylamide or a partially hydrolyzed derivative thereof, a poly(N—(C₁₋₆ alkyl)(meth)acrylamide), a poly(N,N-di(C ₁₋₆ alkyl)(meth)acrylamide, 2-(meth)acrylamido-2-methylpropane sulfonic acid or an alkali salt thereof, or a combination thereof.

11. A method for the manufacture of the composition of claim 10, the method comprising contacting the extractant and the polymeric adsorbent in a solvent under conditions effective to provide the composition.

12. The method of claim 11, further comprising precipitating a water-insoluble polymer on a surface of the composition to provide the composition of claim 10.

13. The method of claim 11, further comprising:
precipitating a water-soluble polymer on a surface of the composition; and
cross-linking the water-soluble polymer on the surface to provide the composition of claim 10.

14. A device comprising the composition of claim 1.

15. The device of claim 14, further comprising at least one secondary adsorbent, wherein the at least one secondary adsorbent is activated carbon, silica, modified silica, a second polymeric adsorbent, or a combination thereof.

16. A method for separating an analyte from a solution, the method comprising:
contacting the composition of claim 1 with the solution to form an analyte-bound composition; and
separating the solution from the analyte-bound composition to provide a regenerated solution,
wherein the amount of the analyte in the regenerated solution is less than the amount of the analyte in the solution.

17. The method of claim 16, further comprising contacting a solvent with the analyte-bound composition, wherein at least a portion of the analyte is removed from the analyte-bound composition and into the solvent.

18. The method of claim 16, wherein one or more of the contacting or separating are performed in the device of claim 14.

19. A hemodialysis or hemofiltration system for using the method of claim 16, wherein the solution is a dialysate and wherein the analyte is urea, creatinine, uremic acid, or a combination thereof.

20. The system of claim 19, further comprising a first analyte sensor to determine the concentration of the analyte in the dialysate and a second analyte sensor to determine the concentration of the analyte in the regenerated dialysate.
